(19) United States
(12) Patent Application Publication
(10) Pub. No.: US 2025/0221762 A1
(43) Pub. Date: Jul. 10, 2025

(54) Methods and Devices for Non-Thermal Treatment of Endometriosis

(71) Applicant: J3 MEDICAL, LLC, Denver, CO (US)

(72) Inventors: Gary LONG, Cincinnati, OH (US); William BOWERS, Westminster, CO (US)

(73) Assignee: J3 MEDICAL, LLC, Denver, CO (US)

(21) Appl. No.: 18/850,915

(22) PCT Filed: Mar. 28, 2023
(86) PCT No.: PCT/US2023/016548

§ 371 Date: Sep. 25, 2024

Related U.S. Application Data
(60) Provisional application No. 63/362,086, filed on Mar. 29, 2022.

(51) Int. Cl. A61B 18/14; A61B 18/00

(52) U.S. Cl. CPC A61B 18/1485; A61B 2018/00559; A61B 2018/00613; A61B 2018/00767; A61B 2018/1467

(57) ABSTRACT

Methods and devices for non-thermal treatment of endometriosis. The non-thermal treatment of endometrial lesions results in electroporation of the endometrial cells, and ultimately necrosis of the lesion. Lesions are identified and electric field parameters determined to substantially remove all of the endometrial cells in the target lesion. Application of the electric field to the lesion creates a treatment zone where the cells necrose, leaving the cellular matrix mostly undisturbed. Devices include a generator capable of applying an electric field to an endometrial lesion through one or more electrodes.

### CROSS-REFERENCE TO RELATED APPLICATIONS

This application claims priority to co-pending U.S. Provisional Patent Application Ser. No. 63/362,086, filed Mar. 29, 2022, the entire disclosure of which is incorporated herein by reference.

### FIELD OF THE INVENTION

The invention relates to non-thermal treatments of endometriosis and more particularly to methods and devices for electric field treatment and removal of endometrial lesions in a subject in need thereof.

### BACKGROUND OF THE INVENTION

Endometriosis is a condition caused by the growth of endometrial cells outside of their normal environment, the uterus. For this condition, endometrial cells, which typically thicken and break-down in the uterus during a female's menstrual cycle, are found in other tissues, for example in the ovaries, fallopian tubes and pelvis. These cells, although not in the uterus, continue to function (thicken and then break-down) and respond to hormonal stimulus as if they are within the uterus, resulting in pain and other symptoms.

It is estimated that about 3 million new cases of endometriosis occur each year, particularly in women between the ages of 25 and 40, women who have a family history of endometriosis without children, and women who suffer unusual menstrual cycles.

Endometriosis can cause pelvic cramping and pain, lower back pain, abdominal pain, excessive menstrual bleeding, fatigue, possible problems with fertility, and has been associated with a higher likelihood of developing ovarian cancer. These symptoms of the condition can be fairly mild in nature, or be more severe in nature, resulting in treatment options that range from over-the-counter pain medications to surgical intervention.

Conventional treatment options, as noted above, depend on the severity of the endometriosis and the patient's symptoms. A number of non-invasive treatments depend on interrupting the natural hormonal cycle of the effected woman, including hormonal therapies, GnRH medications, oral contraceptives, and the like. These treatments limit the natural process of ovulation and thereby prevent the break-down of the endometrial tissue outside the uterus (the source of much of the pain). In more severe cases, a surgical intervention is necessitated for removal of endometrial tissue, particularly where the tissue has caused a significant lesion. In the most severe cases, hysterectomy with or without bilateral salpagooophorectomy is called for, thereby removing some, if not all of the offending tissue. More typically, endometrial lesions are removed through the use of laser ablation, where the offending tissue is heated to removal and the surrounding tissue and blood vessels sealed.

These thermal ablation surgical solutions often result in heat induced damage to the surrounding healthy tissue, which then leaves scarring/fibrosis where the lesion has been removed.

These heat induced damages can have a negative impact on the subject's fertility, particularly if the treatment involves the ovaries.

Against this backdrop, the present disclosure is provided.

### SUMMARY

Embodiments in accordance with the present invention include devices and methods for the non-thermal treating of endometriosis. Embodiments are a significant improvement over conventional treatments that rely upon application of heat to ablate offending cells. The non-thermal treatment of endometrial lesions results in electroporation of the endometrial cells, and ultimately necrosis of the lesion. Unlike thermal treatments, non-thermal treatments avoid scarring and damage to non-lesion cells.

Devices include a generator capable of applying an electric field to an endometrial lesion through one or more electrodes. In some aspect, two electrodes can be inserted into the lesion and are spaced from each other at between 1 mm and 50 mm, and more typically 2 to 20 mm. A voltage of from about 500 to about 3,000 volts is typical for treatment of a lesion, pulsed for from about 25 to about 200 μs. Total number of pulses are determined by effect on the target lesion, but can be from one to ten or more. Generated electric fields form non-thermal treatment zones that cause electroporation of the cells within the zone. Cellular matrix remains undamaged.

Methods include treatments of endometrial lesions in need of non-thermal removal. Lesions are identified and electric field parameters determined to substantially remove all of the endometrial cells in the target lesion. Application of the electric field to the lesion creates a treatment zone where the cells necrose, leaving the cellular matrix mostly undisturbed.

### BRIEF DESCRIPTION OF THE DRAWINGS

FIG. 1 illustrates treatment jaws situated on an illustrative endometrial lesion in accordance with embodiments described herein.

FIG. 2 illustrates a thermal treatment of the same endometrial lesion using conventional treatments, note particularly the 460 C at the edge of the treatment zone.

FIG. 3 illustrates a treatment zone in accordance with an electric field in accordance with embodiments herein.

FIG. 4 is an alternative monopolar electrode in accordance with embodiments herein showing an illustrative treatment zone. At least one of the electrodes may be placed in a body cavity such as a uterine manipulator or a rectal probe. The is will contain the electric field in the body, unlike an RF return pad which is connected to the skin and requires capacitive coupling through the skin for ionic current flow.

FIG. 5 is a flow chart illustrating a method for treating endometriosis in accordance with embodiments herein.

FIG. 6A shows a pair of electrodes having a 10 mm spacing, each electrode having a 0.43 mm diameter. FIG. 6B shows the same pair of electrodes placed into a sweet potato.

FIG. 7 shows use of toluidine blue to clarify damaged cell membranes caused by the embodiments herein. FIG. 7A shows untreated cells and FIG. 7B shows treated cells.

FIG. 8 shows stained treatment zones at zero (A), three hours post treatment (B) and twenty-three hours post treatment (C).

FIGS. 9A, 9B and 9C show electric field lines corresponding to the treatments in FIGS. 8A, B and C.

FIG. 10 is a schematic showing recorded voltage and current during a treatment.

FIG. 11A shows a bipolar grasping device for generating electric fields in accordance with embodiments herein. The device is shown placed into a hole in a potato (B), and corresponding damage to the cells (C) and added isolines in FIG. 7D.

### DETAILED DESCRIPTION

Embodiments in accordance with the present invention include devices and methods for the non-thermal treatment of endometriosis. Aspects of these embodiments rely on delivering an electric field sufficient in size and strength to cause electroporation of the treated cells, but not result in damage to the connective tissue (or matrix) that surround and separate the endometrial cells. This unique delivery of an electric field, in the absence of thermal ablation, causes necrosis of the endometrial cells but limits scarring (and damage to matrix), major benefits of the present embodiments.

In one embodiment, a device for treatment of endometrial lesions is provided. The device is capable of delivering an electric field to the lesion such that a treatment zone is produced. Each treatment zone has an electric field threshold edge that can be expanded or contracted based on the voltage applied by the device. In some aspect the electrodes for delivering the electric field are spaced from 1 to 50 mm apart, and in more typical aspects, from 2 to 20 mm apart. A voltage of between 500 to 3,000 volts is typical. An iso-surface is created. In some aspect, the electric field is delivered over a pulse length of 25 to 200 μs, and more typically 75 to 125 μs. Multiple pulses can be applied, for example, 1-20 pulses or more typically 5-15 pulses. A resting period occurs between pulses, which is used for recovery of the tissue's impedance. Application of a treatment is continued until the application causes necrosis of the endometrial lesion.

In another embodiment, methods for treating a subject having endometriosis are provided. Methods include identifying or determining whether a subject has endometriosis, determining the location, size and volume of the subject's endometrial lesions, and determining the strength and location of an appropriate electric field necessary to non-thermally treat the lesion or lesions. In some aspect a follow-up determination is made to ensure that the lesion or lesions have been removed.

### Endometriosis:

Endometriosis is a condition where endometrial tissue grows and proliferates outside its normal environment, the uterus. As such, endometriosis is characterized by endometrial tissue located, for example, in the ovaries, fallopian tubes, and tissues that line the pelvis. In extreme cases, the endometrial tissue can be found outside the woman's pelvis, although this is fairly rare. Although not found in the uterus, the endometrial tissue is still regulated by a woman's normal monthly hormonal cycle, ending with the breakdown of the tissue. Results of the endometrial tissue break-down outside the uterus can result in inflammation, swelling, and scarring to the surrounding and underlying tissue, all of which can result in pain, bloating, bleeding, and other like symptoms.

In cases where the ovaries are involved, infertility can also result, as well as an increased likelihood of developing ovarian cancer.

Approximately 2 to 10% of women between the ages of 25 and 40 have endometriosis. Conventional therapeutics for these women can include analgesics, pain relievers and the like. In more difficult cases, laparoscopy has been used to thermally remove the endometrial lesion and in very difficult cases, hysterectomy (removal of the uterus, fallopian tubes and ovaries). Where laparoscopy is the primary treatment, the removal results in destruction of the endometrial cells and matrix, with subsequent fibrosis and scarring of the damaged area.

Embodiments herein offer an alternative treatment for the removal of endometrial lesions, removal in the absence of endothermal damage. In the present embodiments, electrical fields of appropriate strength are used to introduce electroporation of cell membranes in treatment zones, thereby causing necrosis of the endometrial cells but leaving the intervening matrix intact. This treatment strategy reduced significantly the potential for fibrosis and scarring after lesion removal. The embodiments that follow provide significant improvement to the conventional endometrial lesion treatments.

### Electric Field Treatment:

Embodiments in accordance with the present invention utilize a flow of electricity to produce a predetermined electric field having a strength useful in the treatment of endometrial lesions. The voltage, gap between electrodes, depth of electrodes into lesion, and shape of electrodes determines the effectiveness of the electric field for any particular lesion. Each of these parameters can be modified to match the shape and size of a particular lesion with a resultant electric field. Resultant electric fields can then be used to substantially to completely remove treated lesions.

In one embodiment, parallel two-needle electrodes are placed between 1 to 50 mm apart in the lesion to generate a treatment zone for a particular lesion. Distance between electrodes is determined by the shape of the lesion, so distances of 1 to 50 mm, 5 to 25 mm and 1 to 10 mm are expected. Depth of electrode insertion into the lesion is determined by the volume of the lesion, where electrodes can be inserted up to half the depth of the lesion, i.e., if the lesion is 20 mm in thickness, the electrodes would be placed up to 10 mm into the lesion. Electrode materials can include stainless steel, platinum, platinum/iridium, and the like. Electrode diameters can be varied but are typically between 1 to 4 mm in external diameter.

In typical embodiments a voltage of between 500 to 3,000 volts can be pulsed into the electrodes, causing the electric field. Pulses can last from between 0.5 to 50 μs, and more typically, 0.8 to 1.25 μs, with an appropriate resting period (˜75-150 ms, necessary to recover the tissues impedance). In some aspect the user performs an appropriate number of pulses to result in the necrosis of substantially all of the cells in the endometrial lesion. Input of voltage can be modified with pulse number and electrode placement to ensure that substantially all of the endometrial cells have been electroporated during a treatment. For example, in some aspect the treatment may only require one pulse, in others 2 pulses with appropriate resting time between. In still others 3 pulses, 4 pulses, 5 pulses, 6 pulses, 7 pulses, 8 pulses, 9 pulses, 10 pulses or 11 or more pulses can be applied to a lesion (each with an appropriate resting time between voltage applications).

As such, a particular treatment zone corresponds to a lesion which then is developed by the appropriate electric field to cause necrosis of the cells by electroporation, i.e., introducing small pores in the cell membranes of the cells in the treatment zone. There is little to no residual heating of the cells. The small pores in the cells ultimately cause necrosis within a short time frame without damaging the matrix of the cells, i.e., no scarring.

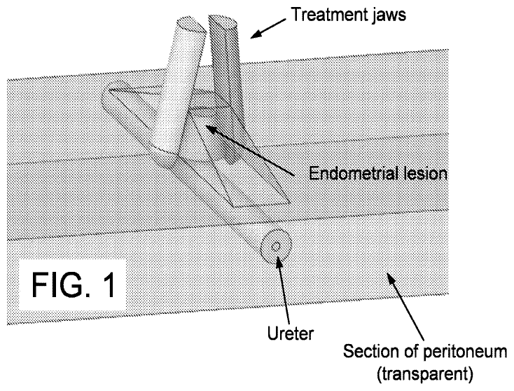

In an alternative embodiment, two electrodes can be placed in a treatment jaw configuration, as shown in FIG. 1. For context, a cross-section of peritoneum is shown with a ureter and endometrial lesion extending upward. The shape and length of the two electrodes can be varied, but are shown in this Figure as having a semicircle cross-sectional shape, where the flat side of each electrode sits along the exterior portion of an endometrial lesion. The electrodes are of a sufficient length to abut some or about all of the lesion within the “treatment jaws.” A illustrative treatment zone is shown when the treatment jaw is used in accordance with embodiments herein.

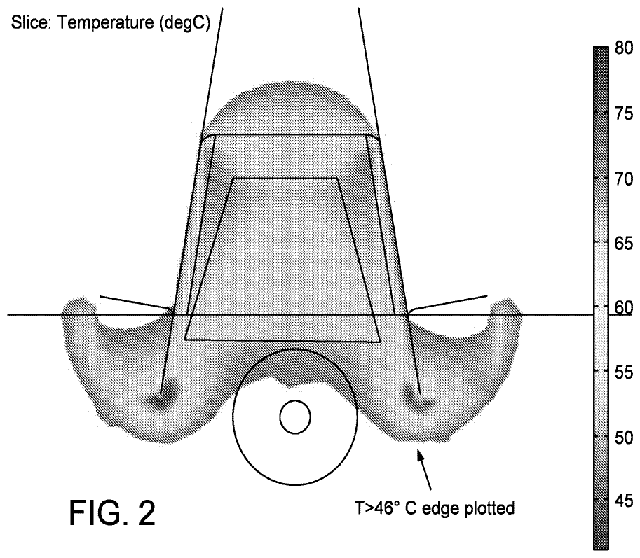

As shown in FIG. 2, the same lesion is treated with thermal ablation. As can be seen from the thermal image, although the lesion is destroyed, the outer edge of the treatment zone reaches a temperature of 460 C. This temperature is sufficient over the period of treatment time to cause clinical damage to the adjacent tissue.

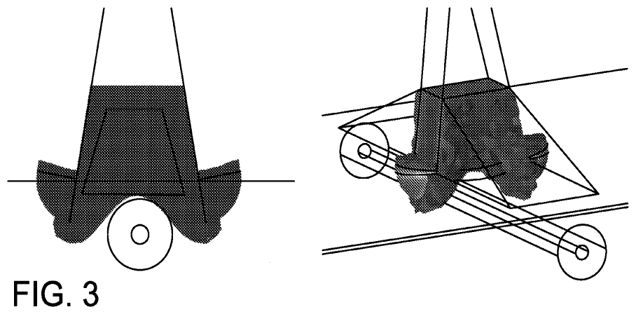

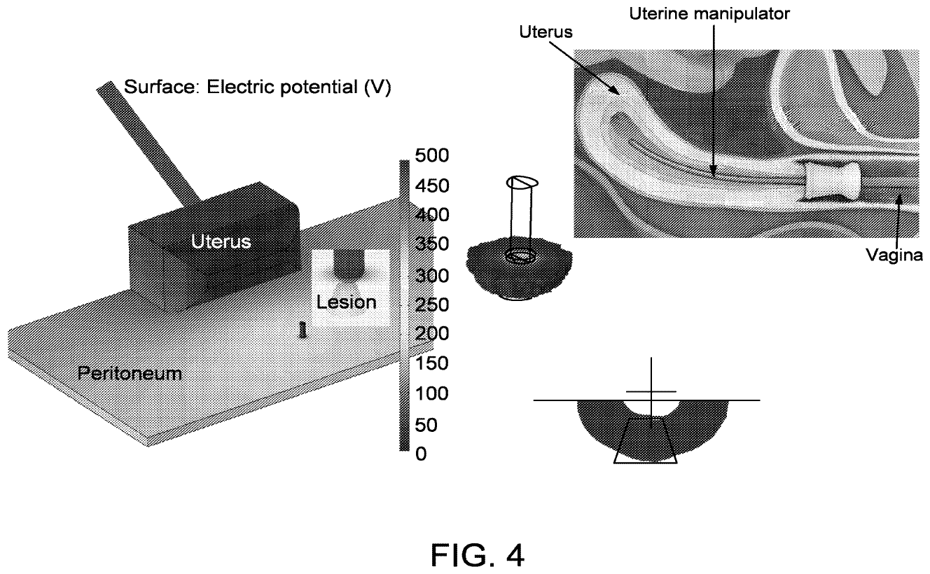

As shown in corresponding FIG. 3, the same lesion treated in accordance with embodiments herein shows little or no thermal heating of adjacent tissue (using bipolar jaws electrodes). In FIG. 4, a monopolar electrode is used to deliver an electric field in accordance with embodiment herein. Again, little or no thermal heading of adjacent tissue is observed. These figures show that use of electric fields to remove endometrial lesions provides a much safer platform than thermal ablation of the same lesion.

As shown in FIG. 4 adaptation, during application of high energy for surgical treatment, a method to safely treat the targeted tissue and return the energy safely to the generator can be accomplished in several different techniques. When utilizing a conventional means, a return pad (RF pad) is used to return the high energy. Conventional methods places a RF pad the patient's skin to complete the RF circuit. The skin contains layers that do not conduct efficiently and results in a capacitive/resistive path through the skin. Using conventional return pad in the described treatment presents difficulties in maintaining the desired squarewave and increases the potential for muscle contractions as the IRE pathways are required to flow through the skeletal muscle.

An improved method is to utilizes a metal probe, such as an uterine manipulator, to return the IRE energy back to the generator without experiencing the problems resulting from using a RF pad. If the metal probe (uterine manipulator) is placed in the uterus, the layers of the uterus are very conductive and improves the conduction path back to the IRE generator. The square wave will remain a square wave and the pulse width is not reduced. As well, the probe in the uterus will reduce the possible pathways through skeletal muscle which reduces the potential for muscle contractions (with an RF pad) and cause muscle contractions. The IRE waveform remains as close to the desired squarewave as possible. This allows the transmembrane voltage applied to the cell throughout the squarewave and results in a lower IRE threshold of treatment.

Electrical fields provide a disruption to the affected cells, causing electroporation of the cellular membrane. The electric field, however, does not cause disruption or destruction of the extracellular matrix. This is a significant advancement in the field as removal of the extracellular matrix leads to fibrosis and scarring, a common side-effect of using conventional therapeutics, like heat ablation.

### Methods of Endometrial Lesion Treatment:

Embodiments in accordance with embodiments herein include methods for non-thermal treatment of endometriosis. In some aspect, a subject is identified in need of the present embodiments. Subjects are typically women aged between 18 and 55 and more typically 25 and 40. Subjects also would typically have endometrial lesions serious enough to require thermal-based laparoscopy or other like conventional treatment. Once a subject has been identified, embodiments of the present invention would be utilized to remove the lesion in replacement of thermal-based laparoscopy.

The identification typically includes both identification of the location of the lesion, the surface area of the lesion, and the depth or volume of the lesion. Lesion identification can be accomplished by MRI or other like methodology.

An electric field of appropriate strength and size is identified for treatment of the subject's endometrial lesion. Such electric fields include identification of a number of parameters, including voltage, number of voltage applications or pulses, electrode material and number of electrodes for use, gap between electrodes, depth of electrodes into lesion, and shape of electrodes as applied to the lesion. In some aspect it is also determined if the electrodes need to be moved after a first treatment to maximum efficiency in eliminating the lesion. Once identified, the treatment parameters are applied to the subject.

In some embodiments, the above treatment zone can be created a second or more times to ensure that all of the endometrial cells have been addressed. In particular, treated cells have been electroporated such that the cells membranes are disrupted, leading to the cells death.

In some embodiments, a follow-up appointment is used to ensure that the electric field was successful in removal of the lesion. Follow-up can include additional applications of appropriate electric fields to the previously treated lesion.

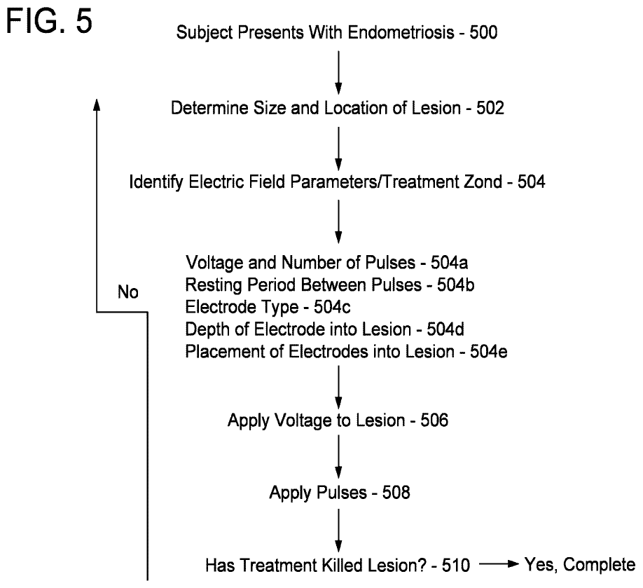

FIG. 5 is a flow chart illustrating one embodiment in accordance with the present invention. In operation 500, a subject presents with endometriosis which requires treatment of endometrial lesions. In operation 502, MRI or other like approach is used to determine the size and location of the lesion. In operation 504, an analysis is performed to identify a set of parameters that would provide a sufficient electric field and thereby treatment zone to cause necrosis of the endometrial cells. In operation 504a voltage and number of pulses is considered, in operation 504b the resting period between pulses is considered, in operation 504c the appropriate electrode type and configuration are considered for delivery of the voltage, in operation 504d the depth to which the electrodes are placed in the lesion is considered, in operation 504e the electrodes are placed in the endometrial lesion as per the previous operation. In operation 506 voltage is applied to create the electric field and thereby treatment zone, In operation 508 application of voltage is pulsed for an appropriate number of times, and in operation 510 a determination is made as to whether the treatment zone has successfully killed or damaged the endometrial cells such that they will necrotic. Optionally, in operation 512, the subject is examined at a later date to confirm removal of the endometrial lesion. If the lesion has not been fully destroyed the process starting at 502 or 504 is repeated through 510 until removal is complete.

The following Examples are for illustrative purposes only and not meant to be limiting in content.

### Examples

EXAMPLE 1: Visualized Treatment Zone for an Applied Voltage The following example shows the results of applying nine different doses to red potato slices. The applied doses illustrate or mimic how tissue is affected by the applied electric field. Pulsed Electric Fields (PEF) are used to cause cell necrosis in live tissue (potato cells). To determine the threshold of cell necrosis potatoes have been used as a tissue phantom. The potato used herein is the red, russet and sweet potatoes. A comparison of treated potatoes in air and stained with Toluidine Blue will be made to assess visualization of the treatment zone.

The difference in each dose is the total on time of the footswitch, which determines the total number of waveforms. We vary the high peak magnitude electric fields to the tissue without enough energy to cause ohmic heating.

The 9 different applications are shown below in Table 1:

TABLE 1
Potatoes as Tissue Phantom Experiments

| | | Set | Cycle | Rest | Packet | Dead | |
|---|---|---|---|---|---|---|---|
| Application | Prototype | Voltage | Count | Period | Count | Time | Waveforms |
| 1 | 10 mm | 1 kV | 10 | 0.01 | 5 | 0.2 sec | 10 |
| | Needle | | | | | | |
| 2 | 10 mm | 1 kV | 10 | 0.01 | 5 | 0.2 sec | 20 |
| | Needle | | | | | | |
| 3 | 10 mm | 1 kV | 10 | 0.01 | 5 | 0.2 sec | 64 |
| | Needle | | | | | | |
| 4 | 10 mm | 2 kV | 10 | 0.01 | 5 | 0.2 sec | 10 |
| | Needle | | | | | | |
| 5 | 10 mm | 2 kV | 10 | 0.01 | 5 | 0.2 sec | 20 |
| | Needle | | | | | | |
| 6 | 10 mm | 2 kV | 10 | 0.01 | 5 | 0.2 sec | 64 |
| | Needle | | | | | | |
| 7 | 10 mm | 3 kV | 10 | 0.01 | 5 | 0.2 sec | 11 |
| | Needle | | | | | | |
| 8 | 10 mm | 3 kV | 10 | 0.01 | 5 | 0.2 sec | 20 |
| | Needle | | | | | | |
| 9 | 10 mm | 3 kV | 10 | 0.01 | 5 | 0.2 sec | 41 |
| | Needle | | | | | | |

In each of the experiments, each does will have a treatment zone containing necrotic cells, and an electric field threshold edge. Voltage can be increased or decreased to vary the zone size. In the experiments, a needle is inserted into the potato and the needle surface boundary condition is set to ground.

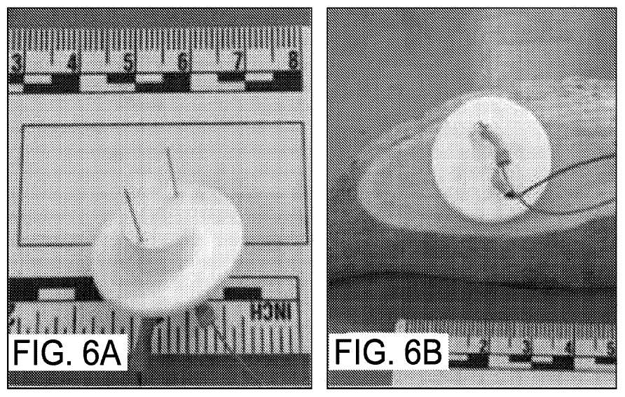

As shown in FIGS. 6A and 6B, needle electrodes having a 0.43 mm diameter and 10 mm spacing (A) are placed into a sweet potato in accordance with the experiments herein (B). FIG. 2 shows the treatment zones in illustrative russet potato 18 hours post treatment. The oxidation of PPO (polyphenol oxidase) in potato results in the visible black electroporation area. The electric field isolines were simulated and placed on the image in photoshop to determine the threshold of treatment.

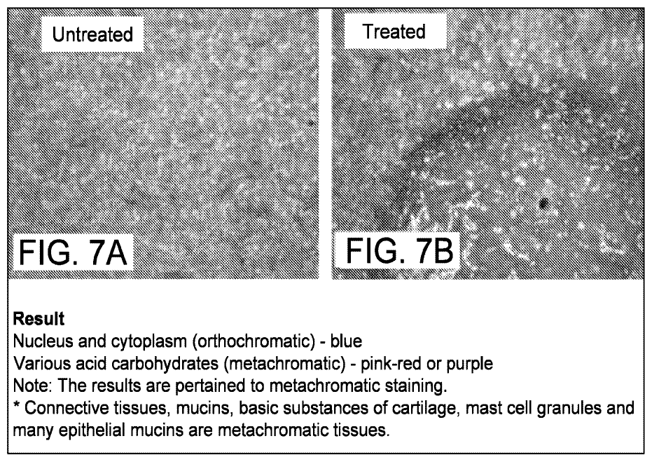

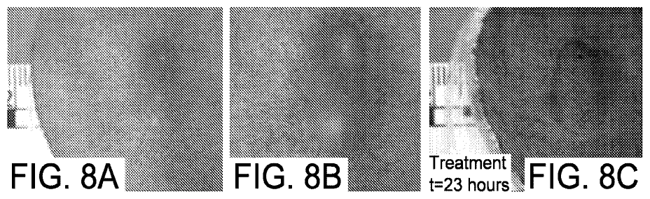

As shown in FIG. 7, toluidine blue powder was mixed in distilled water (0.1 grams/100 ml) to formulate a stain and immediately added to a treated potato to enhance the treatment zone. Treated potato slices at placed treatment side down in the formulated stain for five minutes, followed by a distilled water rinse and then patted dry. The figure shows an untreated and treated russet potato slice. The technique is used to enhance cell walls. A change in color in the treatment zone shows damaged cells as the damaged cells allow infusion of the toluidine blue into the cells. FIG. 8 shows the same experiment over the course of 0 (A). 3 hrs (B) and 23 hrs (C).

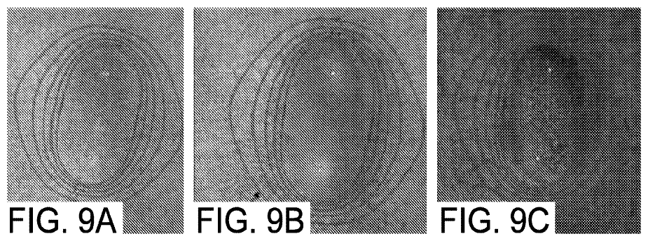

FIG. 9 shows the simulated electric field isolines added to FIGS. 4A, B and C. This Figure shows that at 3 hours you see the minimum time, post staining, where the treatment zone can be identified. The 3 hour zone is about the same as the 23 hour zone.

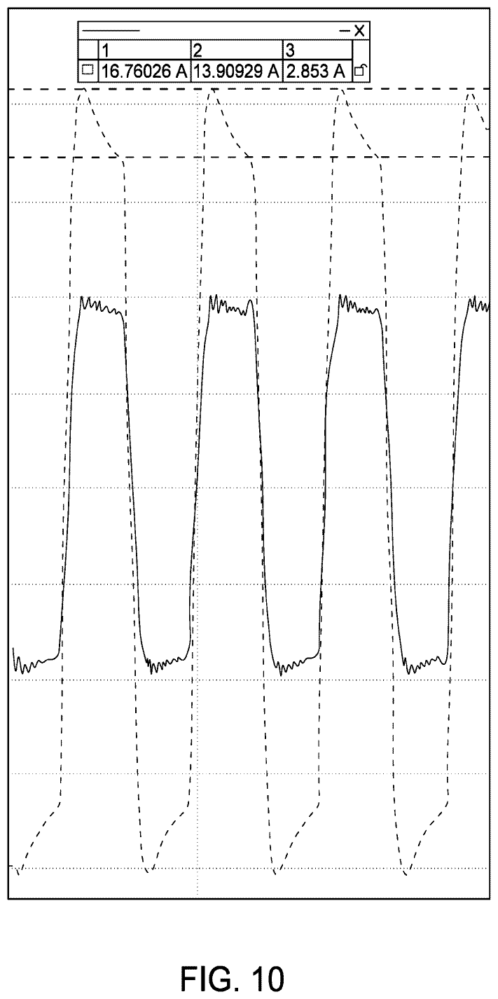

FIG. 10 shows a voltage and current schematic, where the current decreases during a single pulse but can increase up to 50% when multiple pulses are applied, as compared with constant current with a purely resistive load. Similar changes are observed in live animal and human tissue.

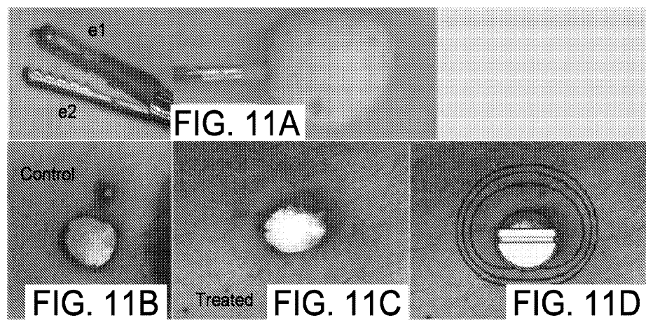

FIG. 11A shows a bipolar grasping device in accordance with the embodiments herein. The grasping device is shown placed into a hole in a potato (11B). An electric field is applied in accordance with embodiments herein and shown in 11C is the dark purple where there is cell damage from boring the hole (as well as a defect in the potato). FIG. 11D shows the treatment zone with isolines places onto the view.

The data shown herein illustrates that potatoes treated with electric fields in accordance with embodiments herein can be used to visualize cell death. Addition of toluidine blue stain to the treated surface is used to enhance the treatment zone. To emulate cellular reaction to electroporation, potatoes cells react in a similar manor to human tissue as the magnitude of the current increases.

What is claimed is:

1. A method of non-thermal treatment of an endometrial lesion in a subject in need thereof, comprising:
a. identifying the location, size and volume of the endometrial lesion in the subject;
b. determining an electric field useful in the treatment of the identified endometrial lesion; and
c. applying the electric field to the endometrial lesion for an appropriate time and voltage to treat the endometrial lesion.

2. The method of claim 1, wherein the determining an electric field includes determining a number of voltage pulses and the width of each pulse in a predetermined time increment.

3. The method of claim 2, wherein the non-thermal treatment of the endometrial lesion causes necrosis of endometrial cells without significant damage to accompanying cell matrix.

4. The method of claim 3, wherein the electric field is provided by a pair of electrodes inserted about 5 mm to 20 mm apart in the endometrial lesion.

5. The method of claim 3, wherein the subject is a woman between the ages of 25 and 50.

6. The method of claim 1, wherein the electric field has a sufficient time and voltage to cause necrosis to cells within the endometrial lesion.

7. The method of claim 6, wherein the electric field does not cause significant damage to the matrix tissue of the endometrial lesion.

8. The method of claim 7, wherein the voltage is from about 500 volts to about 3,000 volts.

9. The method of claim 8, wherein the voltage is applied in two or more pulses lasting 75 μs to about 125 μs.

10. The method of claim 9, wherein the two electrodes are made of stainless steel.

11. A method for generating a treatment zone at an endometrial lesion outside a subject's uterus, comprising:
a. providing one or more electrodes for administering pulsed voltage into the endometrial lesion, wherein the pulsed voltage results in the treatment zone;
b. determining the voltage and number of pulses required to create the treatment zone that substantially eliminates the endometrial lesion;
c. administering the voltage and number of pulses to the endometrial lesion;
d. determining whether additional pulses are required to eliminate the endometrial lesion; and
e. administering any additional pulses to the endometrial lesion.

12. The method of claim 11, wherein the treatment zone causes necrosis of endometrial cells but only has limited effects on extracellular matrix.

13. The method of claim 12, wherein the treatment zone has little to no thermal heating caused by the administering of voltage and number of pulses.

14. The method of claim 12, wherein the one or more electrodes is two electrodes.

15. The method of claim 12, wherein the one or more electrodes is one electrode.

16. The method of claim 14, wherein the two electrodes are placed into the endometrial lesion from about 1 to about 20 mm apart.

17. The method of claim 16, wherein the two electrodes are cylindrical and have two ends, and a first end of each electrode is inserted into the endometrial lesion to half of a depth of the endometrial lesion.
